(19) United States
(12) Patent Application Publication
(10) Pub. No.: US 2017/0266168 A1
(43) Pub. Date: Sep. 21, 2017

(54) METHODS FOR PREVENTING OR TREATING OSTEOARTHRITIS

(71) Applicant: RUSH UNIVERSITY MEDICAL CENTER, CHICAGO, IL (US)

(72) Inventors: Deva Chan, Alexandria, VA (US); Jun Li, Chicago, IL (US); Anna H.K. Plaas, Chicago, IL (US); Daniel Predescu, Chicago, IL (US); John D. Sandy, Chicago, IL (US)

(73) Assignee: RUSH UNIVERSITY MEDICAL CENTER, CHICAGO, IL (US)

(21) Appl. No.: 15/512,325

(22) PCT Filed: Sep. 14, 2015
(86) PCT No.: PCT/US15/49966

§ 371 Date: Mar. 17, 2017

Related U.S. Application Data
(60) Provisional application No. 62/052,206, filed on Sep. 18, 2014.

(51) Int. Cl. A61K 31/4375; A61K 31/551; A61K 31/196; A61K 31/4439; A61K 31/192

(52) U.S. Cl. CPC A61K 31/4375; A61K 31/4439; A61K 31/192; A61K 31/196; A61K 31/551; A61K 2123/00

(57) ABSTRACT

One aspect of the invention provides a method for treating or preventing the development of osteoarthritis by administering to a subject in need of such treatment a composition including a therapeutically effective amount of an anti-fibrotic agent. In various embodiments, the anti-fibrotic agent is 5-methyl-1-phenylpyridin-2-one, tranilast, gamma-glutamyl transpeptidase inhibitor, fasudil, CC-930, T-5524, rosiglitazone, tocilizumab, E5564, TAK-242, GKT136901 or bosentan.

### CROSS-REFERENCE TO RELATED APPLICATIONS

The present patent application claims the benefit of the filing date of U.S. Provisional Patent Application No. 62/052,206, filed Sep. 18, 2014, the contents of which is hereby incorporated by reference.

### STATEMENT AS TO RIGHTS TO INVENTIONS MADE UNDER FEDERALLY SPONSORED RESEARCH AND DEVELOPMENT

This invention was made with Government support of Grant No. R01 AR057066 awarded by the National Institutes of Health. The Federal Government has certain rights in this invention.

### TECHNICAL FIELD

The present invention generally relates to methods for treating or reducing the onset of osteoarthritis. One aspect of the invention provides a method of treating osteoarthritis including administrating an anti-fibrotic agent to a human or veterinary patient in need of such treatment. In one embodiment, the anti-fibrotic agent is 5-methyl-1-phenylpyridin-2-one (“pirfenidone”.)

### BACKGROUND

Increasing degrees of force applied to joints result in joint injury. Such joint injury is frequently seen as a result of trauma, for example chondral lesions are often seen in athletes, and are typically associated with acute inflammation. The treatment of joint injuries (such as ligamentous rupture or meniscal tearing) and rehabilitation of the patient after such injuries involves a number of components. Immediate care after the injury typically includes rest, cold application, compression and elevation. The aim of this treatment is to minimize inflammation, hemorrhage, pain and cellular metabolism during the acute post-injury phase and to optimize the potential for subsequent recovery. In addition, such treatment should prevent the onset of long term damage to the injured tissue. Traumatic injury to joint tissues predisposes to chronic osteoarthritis (OA), which is characterized by synovial fibrosis, subchondral bone remodeling, and severe cartilage erosion.

Such initial treatment is often followed by protection of the injured tissues by immobilization for 1-3 weeks after the injury. Immobilization aims to allow healing to begin and to proceed undisturbed and it also prevents re-injury of the joint which often results in longer recovery times and can have long term effects. After tissue healing has begun, typically beyond 3 weeks post injury, controlled mobilization is introduced. At 4-8 weeks post injury, more vigorous rehabilitation to recover muscle mass and joint function can begin.

Orthopedic repair of severe injuries is often performed as soon as acute swelling and hemorrhage of the injury subsides. However, physicians currently do not have a system or method available to differentiate between acute injury requiring invasive treatment and injuries that will heal sufficiently without such treatment. Traumatic injury to joint tissues predisposes to chronic osteoarthritis (OA), which is characterized by synovial fibrosis, subchondral bone remodeling, and severe cartilage erosion. The acute response to joint injury has been likened to a wound healing cascade of acute inflammation, activation of multipotent repair cells and fibrosis.

### SUMMARY OF THE PREFERRED EMBODIMENTS

In one aspect, the present invention provides a method for treating or inhibiting the development of osteoarthritis. The method includes administering to a subject in need of such treatment a composition including a therapeutically effective amount of an anti-fibrotic agent. The subject may be a human or veterinary subject. In various embodiments, the anti-fibrotic agent is pirfenidone, tranilast, gamma-glutamyl transpeptidase inhibitor, fasudil, CC-930, T-5524, rosiglitazone, tocilizumab, E5564, TAK-242, GKT136901 or bosentan. In one embodiment, the anti-fibrotic agent is pirfenidone. The composition can also include at least one pharmaceutically acceptable carrier.

In one embodiment, the anti-fibrotic agent at least partially normalizes, the activation of at least one gene selected from Col1a1, Col1a2, Col2a1, Col3a1, Acan, Vcan, Has1, Has2, Has3, Itih2 and Tnfaip6. In another embodiment, the anti-fibrotic agent at least partially normalizes the activation of at least one gene selected from the NF-κb pathway genes shown in FIG. 4. In yet another embodiment, the anti-fibrotic agent at least partially normalizes the activation of at least one gene selected from the fibrosis pathway genes shown in FIG. 4.

In one embodiment, the anti-fibrotic agent can be administered orally. In other embodiments, the anti-fibrotic agent is administered by a subcutaneous, intra-articular, intradermal, intravenous, intraperitoneal or an intramuscular route.

In other aspect the method includes determining mRNA expression levels of a plurality of genes expressed in a tissue sample taken from an intra-articular region of a joint of the subject or a blood sample, the plurality of genes comprising at least the genes listed in FIG. 4 and calculating a reparative index score based on the mRNA expression levels of the plurality of genes, wherein the reparative index score is indicative of the quality of the repair response. An anti-fibrotic agent is selected from pirfenidone, tranilast, gamma-glutamyl transpeptidase inhibitor, fasudil, CC-930, T-5524, rosiglitazone, tocilizumab, E5564, TAK-242, GKT136901 and bosentan is administered depending on the reparative index score. In one embodiment, the sample includes synovial fluid, blood, cartilage, synovium, meniscal tissue, joint capsule lining, ligaments or combinations of at least two of these materials.

### BRIEF DESCRIPTION OF THE DRAWINGS

FIG. 1 includes photographs showing India ink macro imaging of joints. Upper—injured WT knee, Middle Injured Has1KO, Lower Injured Has1KO and Pirfenidone.

FIG. 2 are photographs illustrating histology of the intact joint with Safranin-O and Masson's Trichrome stained sections.

FIG. 3 are graphs showing the effect of pirfenidone on selected extracellular matrix gene responses seen with injury to the Has1KO, as compared to the WT cartilage injury model (CIM).

FIG. 4 is a table showing the effect of oral Pirfenidone on elevated gene expression of selected NF-κB and fibrosis pathway genes in Has1KO mice. Genes shown were >2 fold increased over naïve levels at 28 days. Numbers represent the mean of 3 injured joints per treatment type, normalized to 3 naïve, uninjured joints, per genotype.

FIG. 5(A-B) includes graphs showing the effect of pirfenidone on Has1KO joints after injury on arrays of 84 genes in each of the Nf-κB and Fibrosis pathways.

FIG. 6 are photographs illustrating Safranin-O stained Has1KO 4 weeks post-injury. Administration of Pirfenidone reduces the loss of cartilage and subchondral bone in the joint after cartilage injury.

FIG. 7 is a photograph of a Micro-computed Tomography scan showing the effect of pirfenidone administration on bone structure.

FIG. 8 is a photograph of a Micro-computed Tomography scan showing the effect of pirfenidone administration on bone structure of the femur directly posterior to the trochlear groove.

FIG. 9 is a photograph of a Micro-computed Tomography scan showing the effect of pirfenidone administration on bone structure of the cortical bone of the femoral midshaft.

FIG. 10 is a table showing subchondral trabecular bone parameters. Numbers represent the mean of 3 injured joints at day 28, normalized to the mean of 3 uninjured naïve joints, per genotype.

FIG. 11(A-B) are photographs showing Safranin-O (11A) and HA G1-domain (11B) stained histological slides from WT and Has1KO mice. Black arrows indicate synovial hyperplasia; gray arrows indicate cartilage resurfacing; white arrows indicate cartilage loss.

FIG. 12 shows graphs illustrating relative gene abundance (in arbitrary units) in joint tissue compartments. Acan, Vcan V2, Col2a1, Tnfaip6 are affected by loss of Has1.

FIG. 13 shows graphs illustrating NF-κB pathway gene abundance in WT and Has1KO mice after injury

FIG. 14 shows graphs illustrating Fibrosis pathway gene abundance in WT and Has1KO mice after injury

### DETAILED DESCRIPTION OF THE PREFERRED EMBODIMENTS

### Definitions

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”) provided herein, is intended merely to better illuminate the invention and does not pose a limitation on the scope of the invention unless otherwise claimed. No language in the specification should be construed as indicating any non-claimed element as essential to the practice of the invention.

The term “therapeutic effect” as used herein means an effect which induces, ameliorates or otherwise causes an improvement in the pathological symptoms, disease progression or physiological conditions associated with or resistance to succumbing to a disorder, for example restenosis, of a human or veterinary patient. The term “therapeutically effective amount” as used with respect to a drug means an amount of the drug which imparts a therapeutic effect to the human or veterinary patient.

The term “normalize” as used herein to refer to the action of an agent on the activity of a gene means an effect that returns the activity of the gene to a level indicative of a non-diseased state. The term “at least partially normalizes” refers to the action of an agent that at least partially returns the activity of the gene to a level indicative of a non-diseased state.

### Methods for Preventing or Treating Osteoarthritis

For the purpose of promoting an understanding of the principles of the invention, reference will now be made to embodiments, some of which are illustrated in the drawings, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. In the discussions that follow, a number of potential features or selections of assay methods, methods of analysis, or other aspects, are disclosed. It is to be understood that each such disclosed feature or features can be combined with the generalized features discussed, to form a disclosed embodiment of the present invention.

One aspect of the present invention provides a method of treating or preventing the onset of osteoarthritis. In one aspect, the method includes administering at least one anti-fibrotic agent to a human or veterinary subject in need of such treatment. In certain embodiments, the anti-fibrotic agent is pirfenidone, tranilast, gamma-glutamyl transpeptidase inhibitor, fasudil, CC-930, T-5524, rosiglitazone, tocilizumab, E5564, TAK-242, GKT136901 or bosentan. In one preferred embodiment, the anti-fibrotic agent is pirfenidone.

For example, the present method of treatment can be used to prevent or reduce post-traumatic-osteoarthritis (PTOA) and/or other progressive degeneration of orthopedic tissues after injury. The injury can be a joint-related, soft tissue injury. The joint can be any joint in the body of the human or veterinary subject including, but not limited to, the knee, shoulder, hip, elbow, temporomandibular or ankle joints, or a joint of the hand, foot or spine. In these embodiments, the purpose of such treatment can be the protection of joint tissues from inflammatory, fibrotic, and destructive or otherwise non-reparative changes which occur prior to or during PTOA. In some embodiments, the subject can be treated in the early phases of the pathology with the aim of preventing progressive degeneration and PTOA. For example, the subject may be treated before the onset of the symptoms of osteoarthritis, for example, immediately after a joint injury or after a joint surgical procedure.

The anti-fibrotic agent can be, but need not be, administered in combination with another therapeutic technique. Such additional treatments can include, but are not limited to, surgical reconstruction, physical therapy, viscosupplementation HA therapy, diet recommendations or life-style change recommendations. In other embodiments, the treatment includes administration of anti-inflammatory creams, gels or sprays, heat and freeze treatments, non-steroidal anti-inflammatory drugs (NSAIDs), acupuncture, complementary and alternative medicines, steroid injections or steroid tablets.

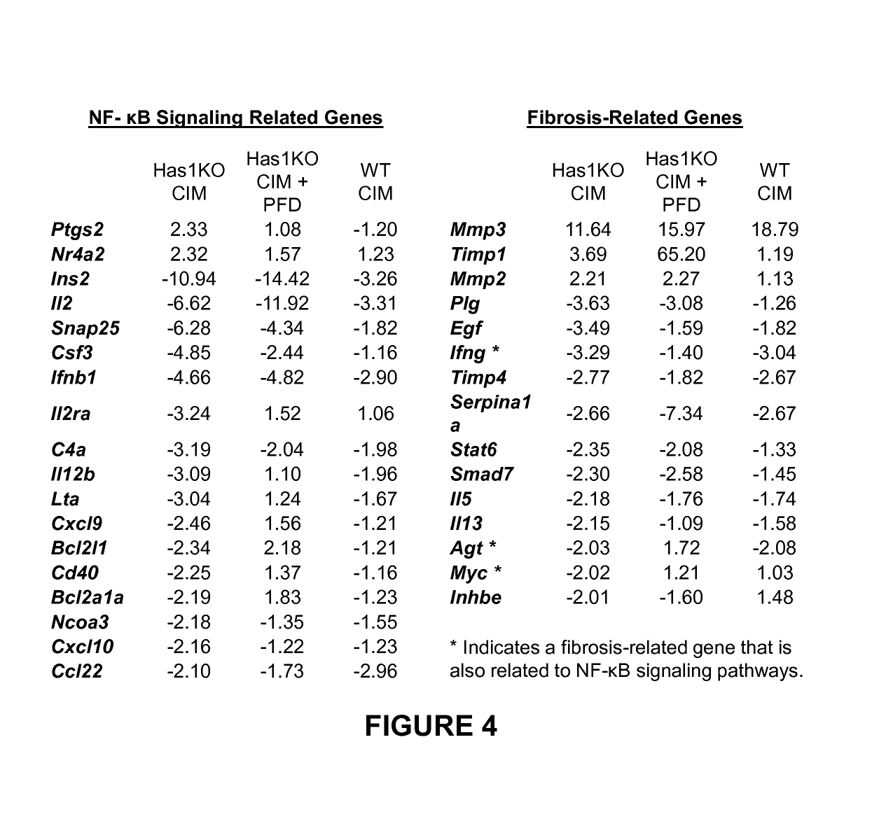

The anti-fibrotic agent can act by exhibiting inhibitory effects on the expression of pro-inflammatory and pro-fibrotic genes. In some embodiments, the anti-fibrotic agent is delivered in an amount sufficient to reduce the activation of at least one gene from the NF-κb pathway genes shown in FIG. 4. In other embodiments, the anti-fibrotic agent at least partially normalizes the activation of at one gene from the fibrosis pathway genes shown in FIG. 4. In yet other embodiments, the anti-fibrotic agent modifies the activation of 2, 3, 4, 5, 6, 7, 8, 9, 10, 11, 12, 13, 14, 15, 16, 17, 18, 19, 20, or 21 of the NF-κb pathway genes shown in FIGS. 4 and/or 2, 3, 4, 5, 6, 7, 8, 9, 10, 11, 12, 13, 14, or 15 of the fibrosis pathway genes shown in FIG. 4. In yet other embodiments, the anti-fibrotic agent at least partially normalizes the activation of at least 1, 2, 3, 4, 5, 6, 7, 8, 9, 10 or 11 of the Col1a1, Col1a2, Col2a1, Col3a1, Acan, Vcan, Has1, Has2, Has3, Itih2 or Tnfaip6 genes.

### Pharmaceutical Compositions

Another aspect of the present invention provides pharmaceutical compositions including at least one of the anti-fibrotic agents disclosed above. For example, the pharmaceutical composition may include 1, 2, 3, 4, 5 or more of such anti-fibrotic agent(s). The pharmaceutical compositions can be in the form of, for example, tablets, pills, dragees, hard and soft gel capsules, granules, pellets, aqueous, lipid, oily or other solutions, emulsions such as oil-in-water emulsions, liposomes, aqueous or oily suspensions, syrups, alixiers, solid emulsions, solid dispersions or dispersible powders. In pharmaceutical compositions for oral administration, the agent may be admixed with commonly known and used adjuvants and excipients, for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. GELUCIRE). In the pharmaceutical composition, the agent may also be dispersed in a microparticle, e.g. a nanoparticulate, composition.

For parenteral administration, the agent or pharmaceutical compositions of the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally, for parenteral administration the agent or pharmaceutical compositions of the agent can be in the form of an aqueous, lipid, oily or other kind of solution or suspension or even administered in the form of liposomes or nano-suspensions.

### Modes of Administration

The anti-fibrotic agents or pharmaceutical compositions including the anti-fibrotic agents can be administered by any method that allows for the delivery of a therapeutic effective amount of the agent to the subject. Modes of administration can include, but are not limited to, oral, topical, transdermal and parenteral routes, as well as direct injection into a tissue, for example a joint, and delivery by a catheter. Parenteral routes can include, but are not limited to subcutaneous, intradermal, intra-articular, intravenous, intraperitoneal and intramuscular routes. In one embodiment, the route of administration is by topical or transdermal administration, such as by a lotion, cream, a patch, an injection, an implanted device, a graft or other controlled release carrier. Routes of administration include any route which directly delivers the composition to the systemic circulation (e.g., by injection), including any parenteral route. Alternatively, administration can be by delivery directly to the affected joint capsule.

One embodiment of the method of the present invention comprises administering at least one anti-fibrotic agent, for example pirfenidone, in a dose, concentration and for a time sufficient to prevent the development of, or to lessen the extent of, osteoarthritis. Certain embodiments include administering systemically at least one anti-fibrotic agent in a dose between about 0.1 micrograms and about 100 milligrams per kilogram body weight of the subject, between about 0.1 micrograms and about 10 milligrams per kilogram body weight of the subject, between about 0.1 micrograms and about 1 milligram per kilogram body weight of the subject. In practicing this method, the anti-fibrotic agent or therapeutic composition containing the agent can be administered in a single daily dose or in multiple doses per day. This treatment method may require administration over extended periods of time. The amount per administered dose or the total amount administered will be determined by the physician and will depend on such factors as the mass of the patient, the age and general health of the patient and the tolerance of the patient to the compound.

Methods for Determining the Quality of Recovery from Joint Injury and the Need for Treatment of Such Injuries

In another aspect of the present invention, the anti-fibrotic agent is administrated dependent on the status of the transcriptome of tissue containing stromal and multipotent progenitor cells proximate to the injury site. For example, a tissue sample can be a sample taken from the intra-articular space of the injured joint or a blood sample. As used herein the term “intra-articular” refers to the space inside of a joint between two or more bones, specifically to the portion of the joint contained by the joint capsule. The sample can include synovial fluid, blood, cartilage, synovium, meniscal tissue, joint capsule lining, ligaments or combinations of at least two of these materials. In one embodiment, the need for the administration of the anti-fibrotic agent is determined on the basis of the valve of a reparative index that is indicative of tissue transcriptome.

Examples of methods of generating such an index are disclosed in co-pending PCT Patent Application Number PCT/US2014/54550, filed Sep. 8, 2014, the contents of which are incorporated by reference. In one embodiment, calculating the reparative index score includes comparing the mRNA expression levels form the subject's tissue sample with first standard expression levels of the plurality of genes and/or second standard expression levels of the plurality of genes. The first standard expression levels are indicative of a reparative profile and the second standard expression levels are indicative of a non-reparative profile.

Other methods of determining the quality of recovery from joint injury may be used in combination with, or instead of, the method disclosed above. Such methods include, but are not limited to, noninvasive imaging, for example micro-computed tomography, and arthroscopy, especially of bone and cartilage structure and quality. For the purposes of this analysis, a reparative profile is considered to a profile indicative of tissue repair without the onset of osteoarthritis. A non-reparative profile is considered to be a profile indicative of the development of osteoarthritis. The reparative index score is based on relative values of the subject's tissue mRNA expression levels, the first standard expression levels and the second standard expression levels.

Embodiments of the invention will be further described in the following examples, which do not limit the scope of the invention described in the claims.

### EXAMPLES

### Example 1—Deficiency of Hyaluronan Synthase 1 (HAS1) Results in Chronic Joint Inflammation and Widespread Intra-Articular Fibrosis in a Murine Model of Knee Joint Cartilage Damage

Under an approved IACUC protocol, a non-bleeding cartilage injury to the patellar groove of 10-12 week male C57Bl6 mice (WT, Has1KO or Has3KO) was followed for 4 weeks. Joint surfaces were macro-imaged after India ink and histologically after Safranin-O and HA staining. For gene expression at 1 and 4 weeks, separated tissues (CSB, MESY, PT) were dissected into RNALater, where CSB was cartilage/subchondral bone from both surfaces, MESY was medial and lateral menisci with synovium, and PT was distal patellar tendon. TAQMAN®-based qPCR was with inventoried probes for the following genes of interest (GOIs): Acan, Col1a1, Col2a1, Col3a1, Gapdh, Has1, Has2, Has3, Itih2, Tnfaip6, Vcan V1 (previously V0) and Vcan V2 (previously V1). mRNA abundance (arbitrary units) was calculated as 1000×2−ΔCt, where ΔCt is Ct(GOI)−Ct(Gapdh).

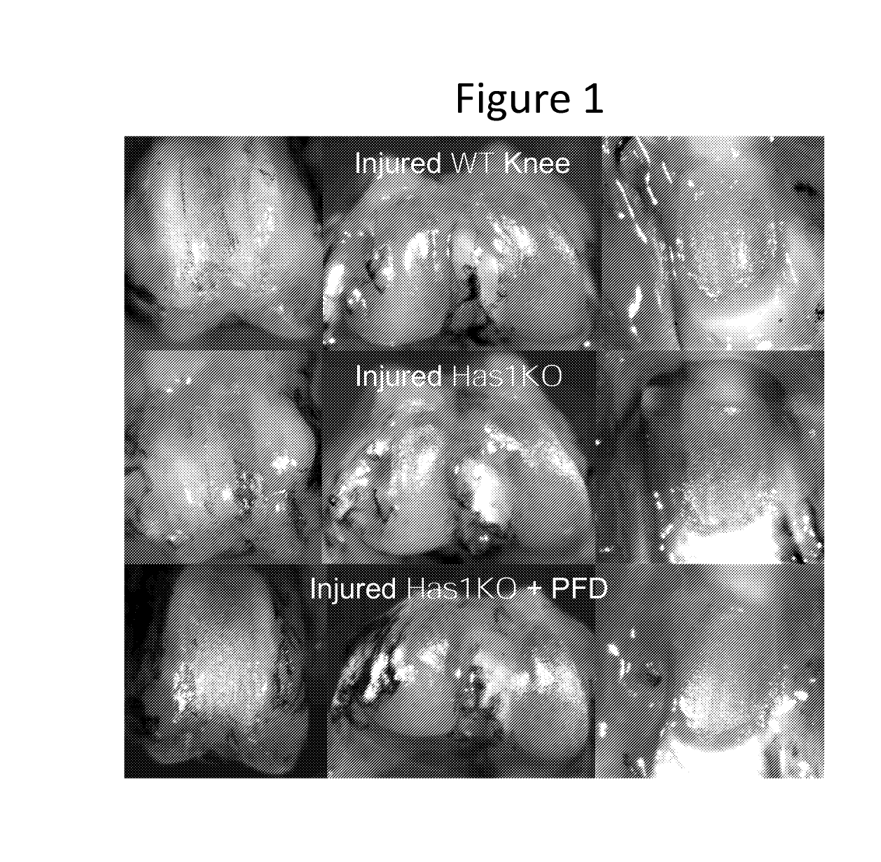

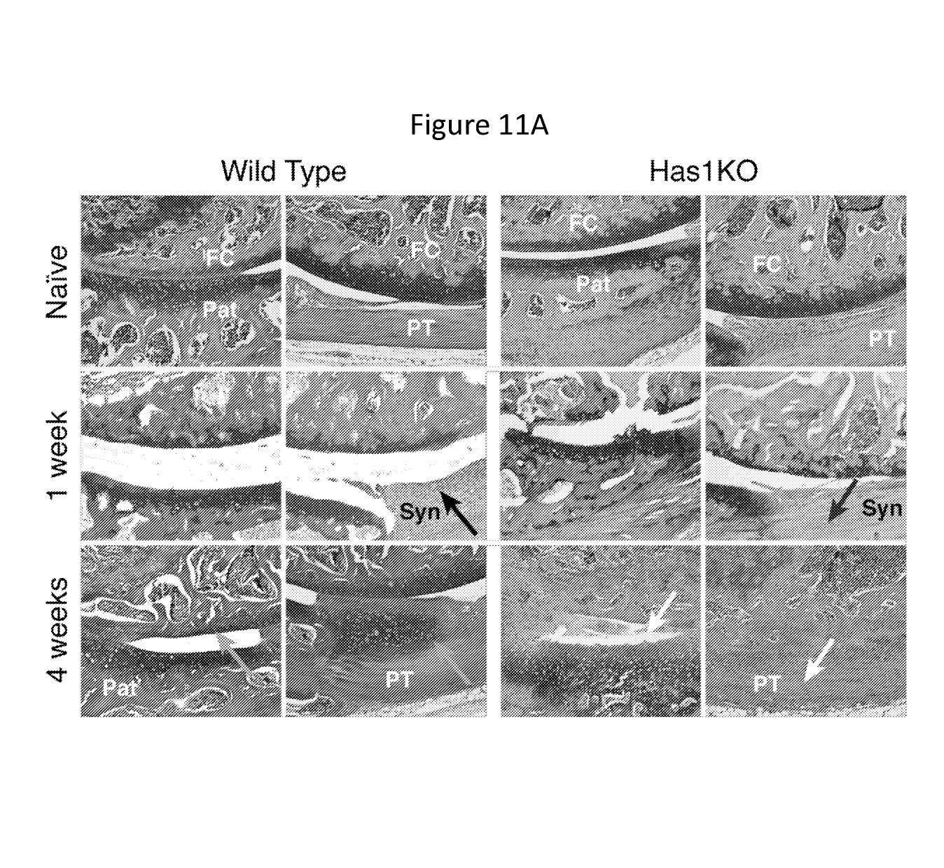

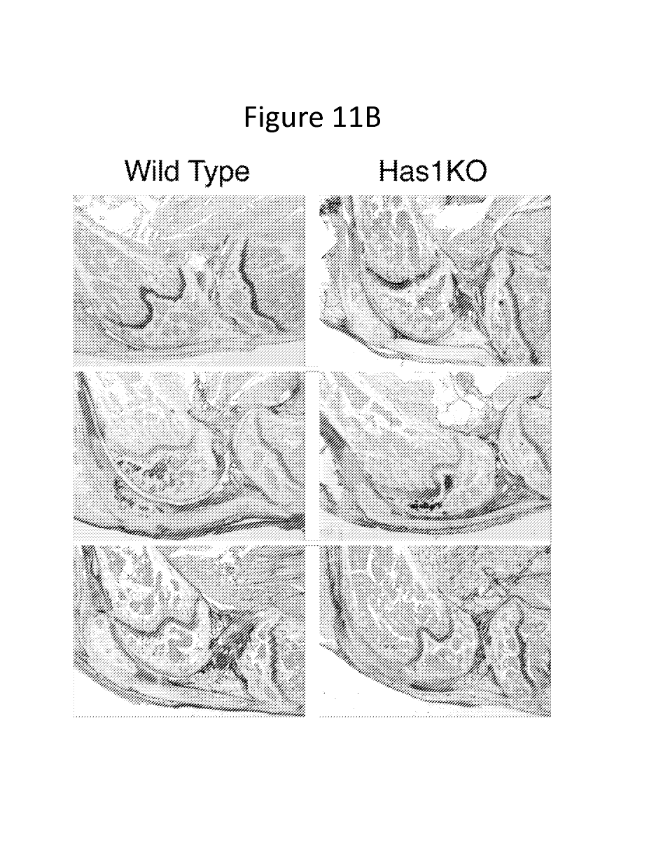

Macroscopic joint imaging and Safranin-O staining of WT joints at 4 week post-injury showed a resurfacing response surrounding the damaged groove cartilage (FIG. 1). There was some wear evident on the apposing patellar cartilage but no detectable damage to femoral or tibial surfaces. This non-destructive WT response was also seen in the Has3KO joints; however, Has1KO mice showed comprehensive changes including chondrophytes at the margins of the cartilage injury (FIG. 1), and cartilage loss from the patella and femoral condyles with fibrosis of synovium and joint capsule. In HA imaging studies (FIGS. 11 A and B), there was an accumulation of HA in meniscus, synovium, tendon, and joint capsule and notably this occurred in both WT and Has1KO mice.

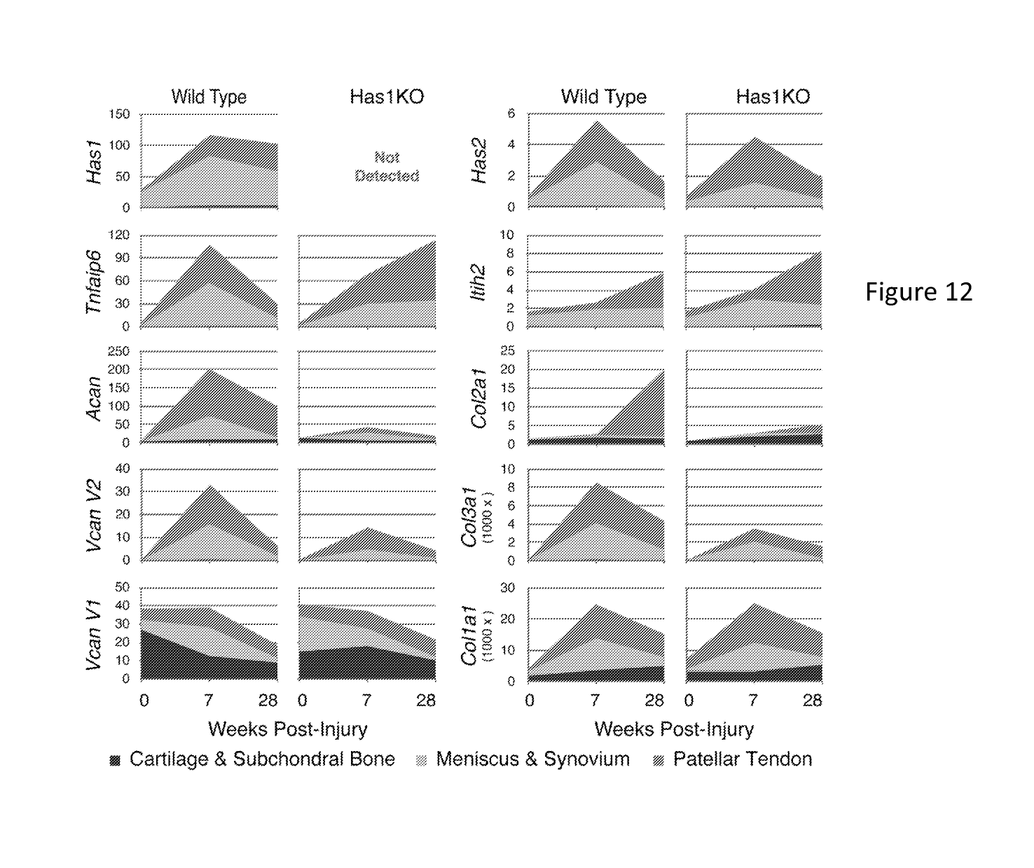

To investigate the mechanism of excessive remodeling in Has1KO mice, KO and WT were compared for expression of the target genes at 0, 1 and 4 weeks post injury in the different tissues, CSB, MESY, and PT (FIG. 12). Deletion of Has1 was accompanied by no (or minor) changes in the time course profile or transcript abundance for Has2, Itih2, Vcan V1 and Col1a1. However, deletion resulted in more pronounced changes in these parameters for Vcan V2 and Col3a1, both of which were reduced ˜3-fold at 1 wk. Most notably, Has1 deletion resulted in ˜10-fold reduction in Acan expression at both 1 and 4 weeks and in Col2a1 at 4 wks. It therefore appears that a generalized reduction (i.e., lack of activation) in expression of both proteoglycans (Acan and Vcan V2) and collagens (Col2a1 and Col3a1) accompanies the aberrant tissue remodeling in Has1KO mice. This suggests that activation of these genes in WT mice is a reparative response but this does not occur in Has1KOs.

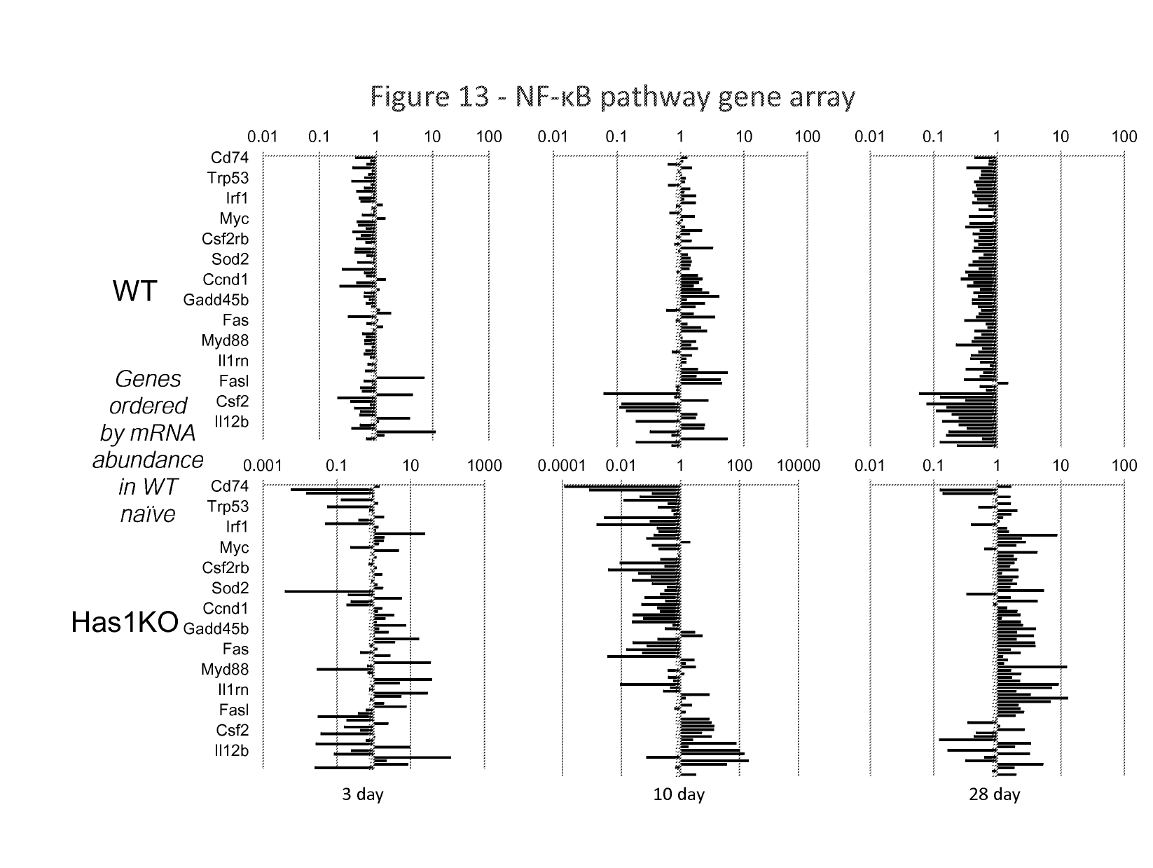

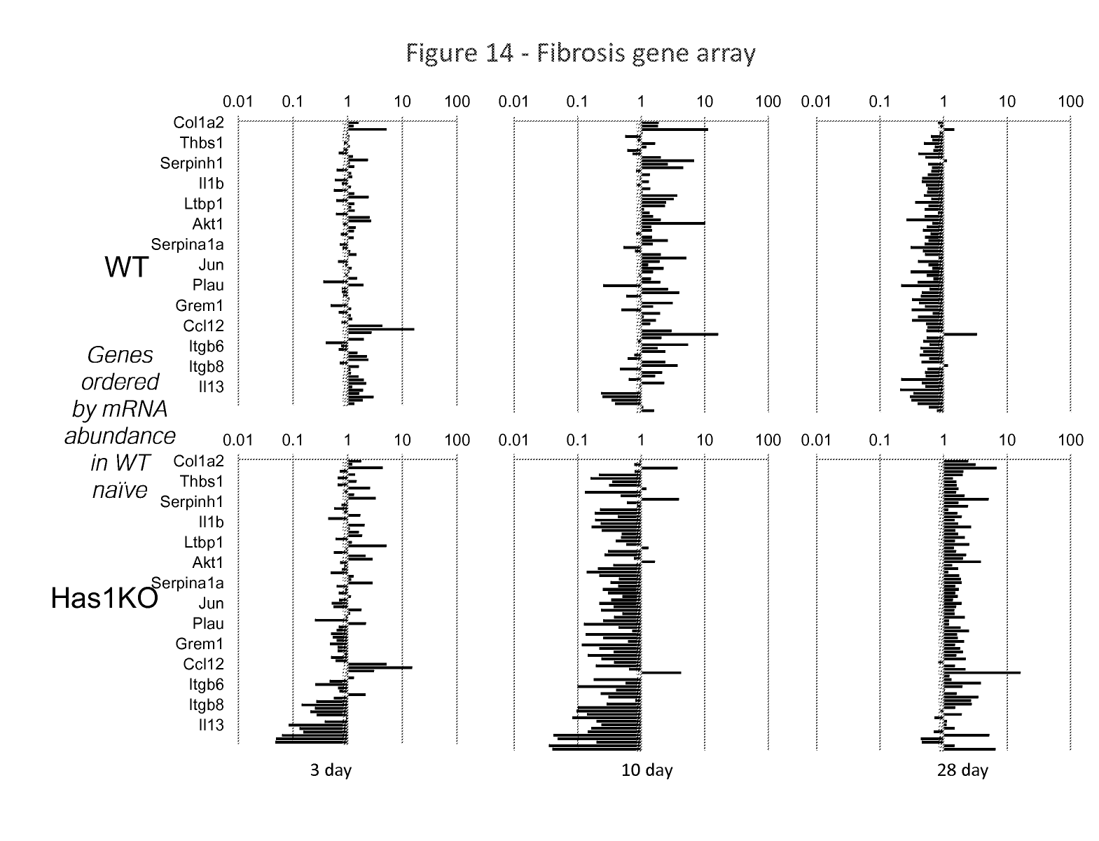

The analysis has also provided novel information on the relative transcript abundance of these genes in the cells of the CSB, MESY, and PT. While naïve levels were generally similar in PT and MESY, most striking was the finding that for both WT and Has1KO mice, all the genes studied here showed an abundance profile at the peak of activation (1 week) of PT>MESY>>CSB (FIG. 13 NF-κB pathway and FIG. 14—Fibrosis pathway.) The high levels in the PT suggest that the cells of this tissue were most responsive to the cartilage injury, possibly due to the fact that that the injury site and PT are in such close proximity, and may experience perturbed biomechanical forces.

Cartilage injury in the proximal femoral groove in WT mice resulted in a transient synovial and joint capsule hyperplasia and patellar tendon swelling, which was accompanied at 1 week by enhanced expression of Has1, Has2, Acan, Vcan V2 and Tnfaip6 (FIG. 12). This is consistent with an accumulation of HA in the ECM of these tissues (FIG. 11B). At 4 weeks post-injury, these expression levels had essentially normalized. Moreover, by 4 weeks, resurfacing at the injury site was seen with no additional cartilage damage on other surfaces, indicative of an effective ‘healing’ response in WT joints (FIG. 11A). Has1KO mice however showed less robust gene responses at 1 week post-injury, and no tissue healing responses were seen histologically at 4 weeks, despite the accumulation of HA (FIG. 11 and FIG. 12). Instead, the synovium and joint capsule had developed extensive fibrotic lesions, accompanied by cartilage loss on adjacent surfaces (FIG. 11A, right side panels). Furthermore, the persistent increase in expression of Tnfaip6 (TSG6) out to 4 weeks post-injury (in all Has1KO tissues, but not in WT), is consistent with the development of a chronic inflammatory environment in the Has1KO joints, leading to bone and cartilage damage, as has been reported by others for human OA and RA.

### Example 2—Evaluation of Cartilage Injury: Effect of Administration of Pirfenidone

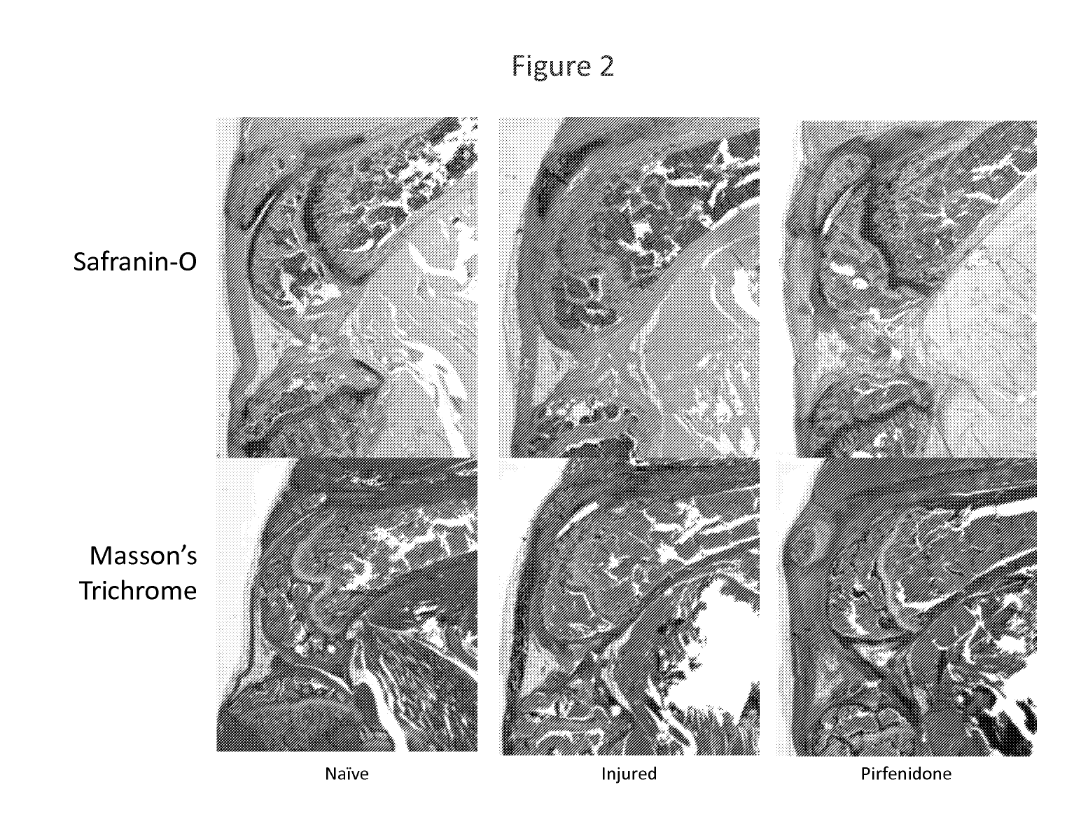

Under approved IACUC protocols, a confined non-bleeding cartilage injury was created in the patellar groove of the right legs of 12-wk old male WT and Has1KO (C57Bl6) mice. Pirfenidone was provided in the chow (5 mg/g, Harlan) starting 3 days after surgery, and mice ingested about 600 mg pirfenidone/kg body weight daily. Joint tissue pathology was evaluated by photography of joint tissue surfaces after India Ink application (FIG. 1) and by histology of the intact joint with Safranin-O and Masson's Trichrome stained sections (FIG. 2.) For gene expression studies, skin and muscle was removed and whole knee joints were stored in RNALater. Inventoried TAQMAN® probes for Gapdh, Has2, Tnfaip6, Col1a1, Col3a1 and Vcan V2 were used to measure expression at 0, 7, 14, and 28 days post-injury, and Qiagen RT2 Profiler PCR Assay mouse arrays for NF-κB (PAMM-225ZA) and Fibrosis (PAMM-120ZA) pathways were used to assay whole joints at 21-28 days after injury, with and without pirfenidone treatment. Relative mRNA abundance was calculated as 1000×2−ΔCt, with ΔCt defined as Ct of the gene of interest minus Ct of Gapdh. Fold change was defined as abundance at a time point divided by abundance in the naïve condition for the same genotype.

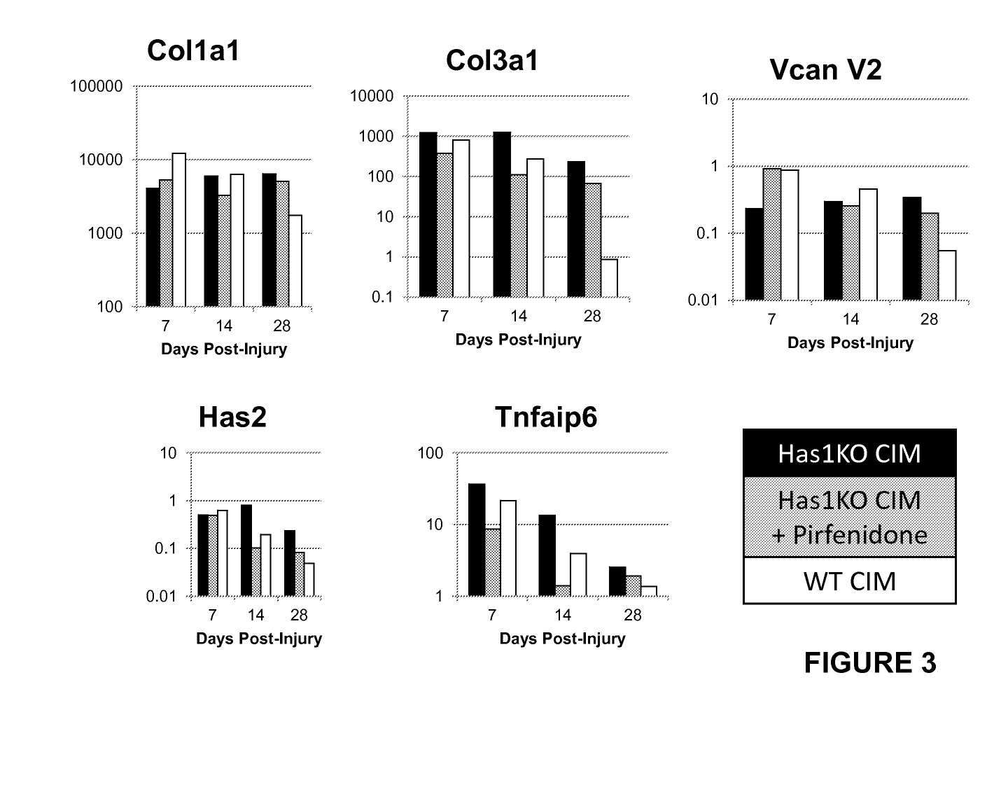

The effect of pirfenidone treatment on whole joint gene expression was examined. These studies showed (FIG. 3) that pirfenidone normalized expression of genes linked to the aberrant wound healing responses seen without pirfenidone in the Has1KO. For example, in Has1KO, Has2 expression was delayed and more prolonged than in the WT, and both of these features were normalized by pirfenidone treatment. Tnfaip6 expression was enhanced at 2 weeks in Has1KO and again normalized by pirfenidone. Notably, Vcan V2 was markedly inhibited in Has1KO, at 7 days, and even this inhibition was corrected by the drug.

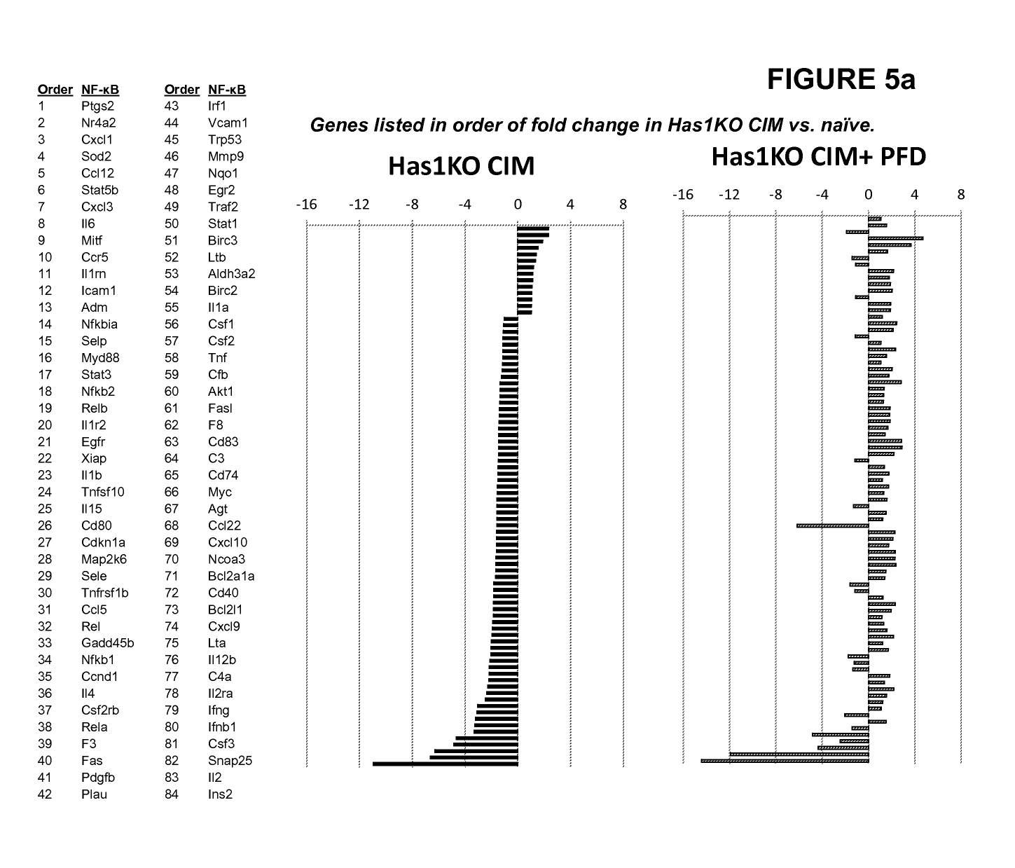

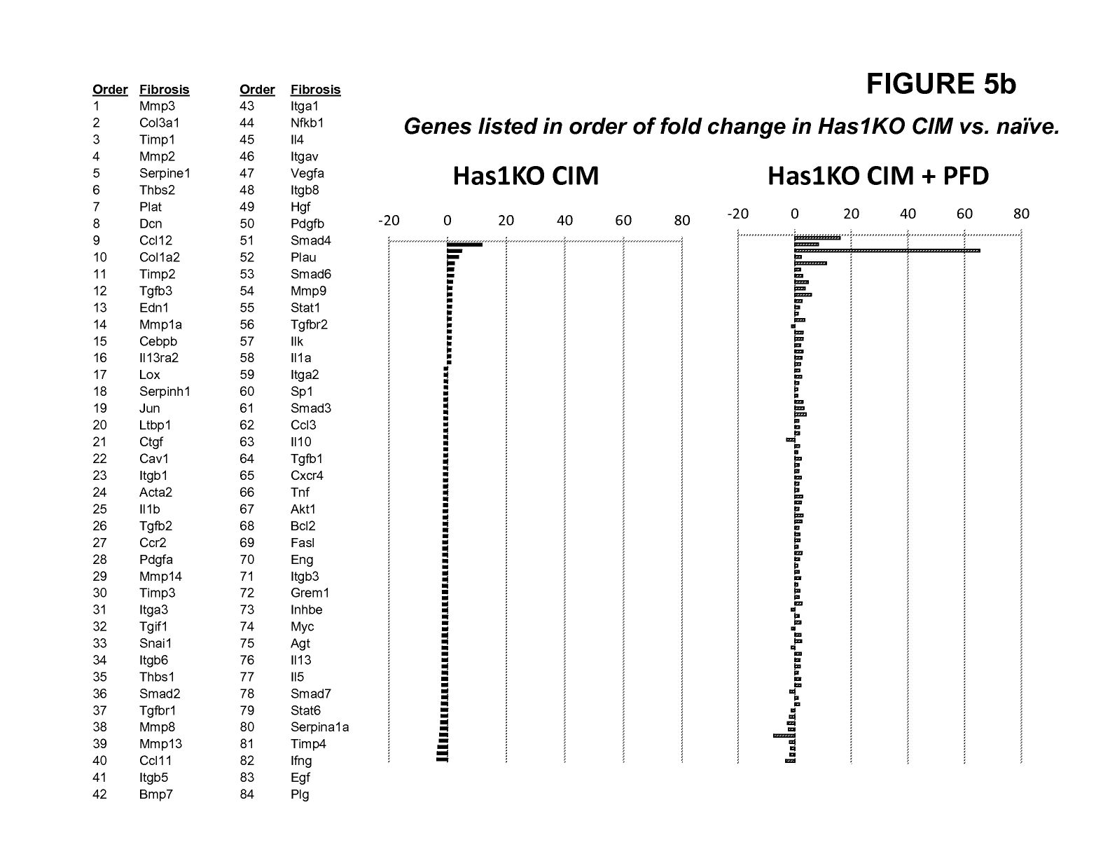

To investigate this apparently therapeutic effect of pirfenidone in more detail, we examined its effect on arrays of 84 genes in each of the Nf-κB and Fibrosis pathways and report here that a 24 day drug treatment post injury has a marked effect on the expression of genes in the two pathways (FIGS. 4, 5A, and 5B). For example, the activation of anti-apoptotic, anti-fibrotic, and matrix remodeling genes, such as Sod2, Ccl12, II13ra2, Serpine1, and Timp1, was enhanced with pirfenidone.

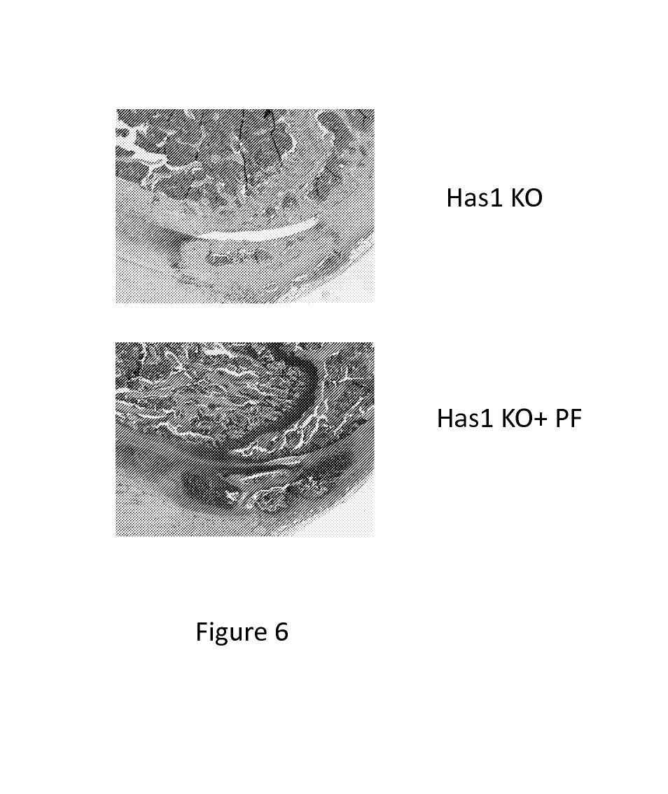

In the same way, the effects on expression of pro-inflammatory and pro-fibrotic genes were accompanied by profound effects on tissue pathology. A histologic examination of sagittal sections across the entire joint was done on 3 mice from each group. In all cases, pirfenidone treatment blocked the appearance of fibrotic tissues, and drug treatment was accompanied by a marked enhancement of Safranin-O staining in the cartilages (including growth plates) and replacement of fibrotic tissue with cartilage-like deposits at the margins of the patella (FIG. 6).

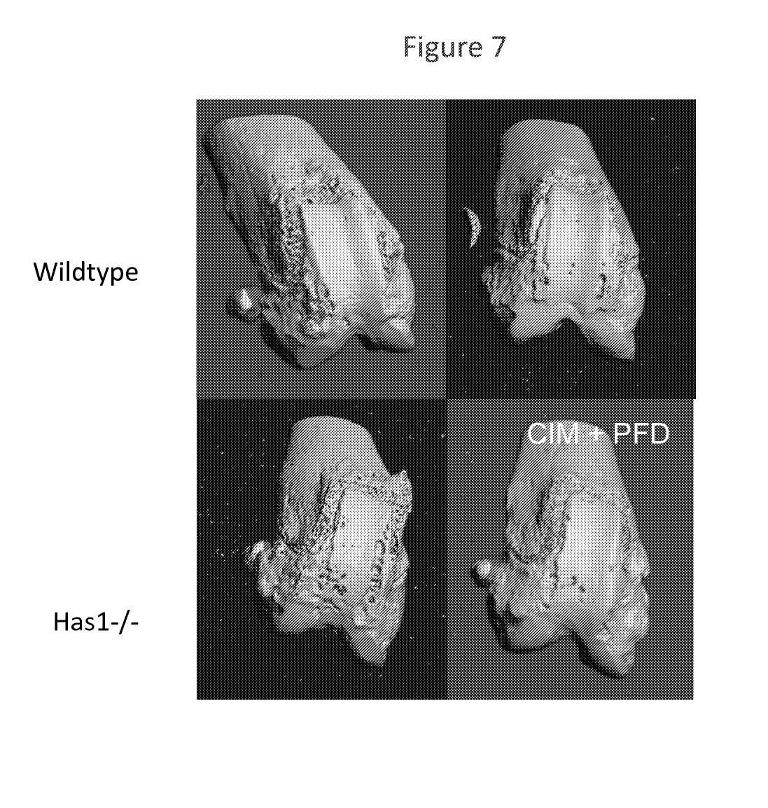

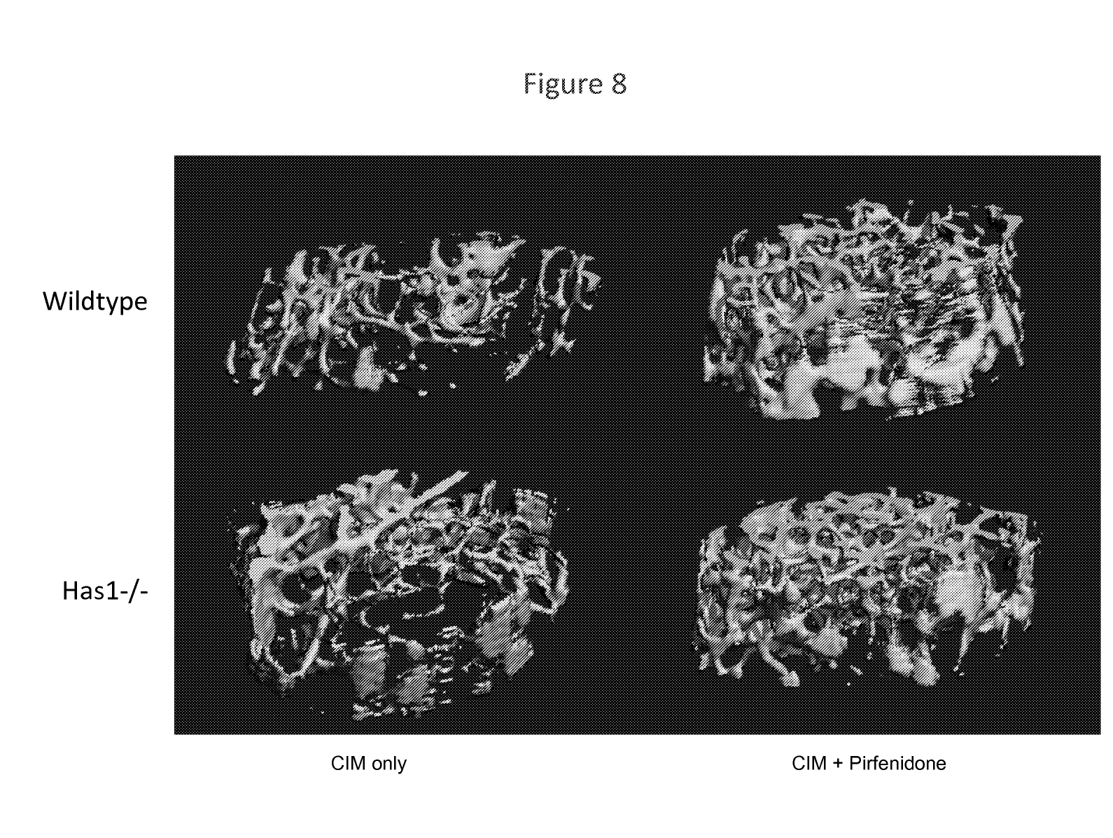

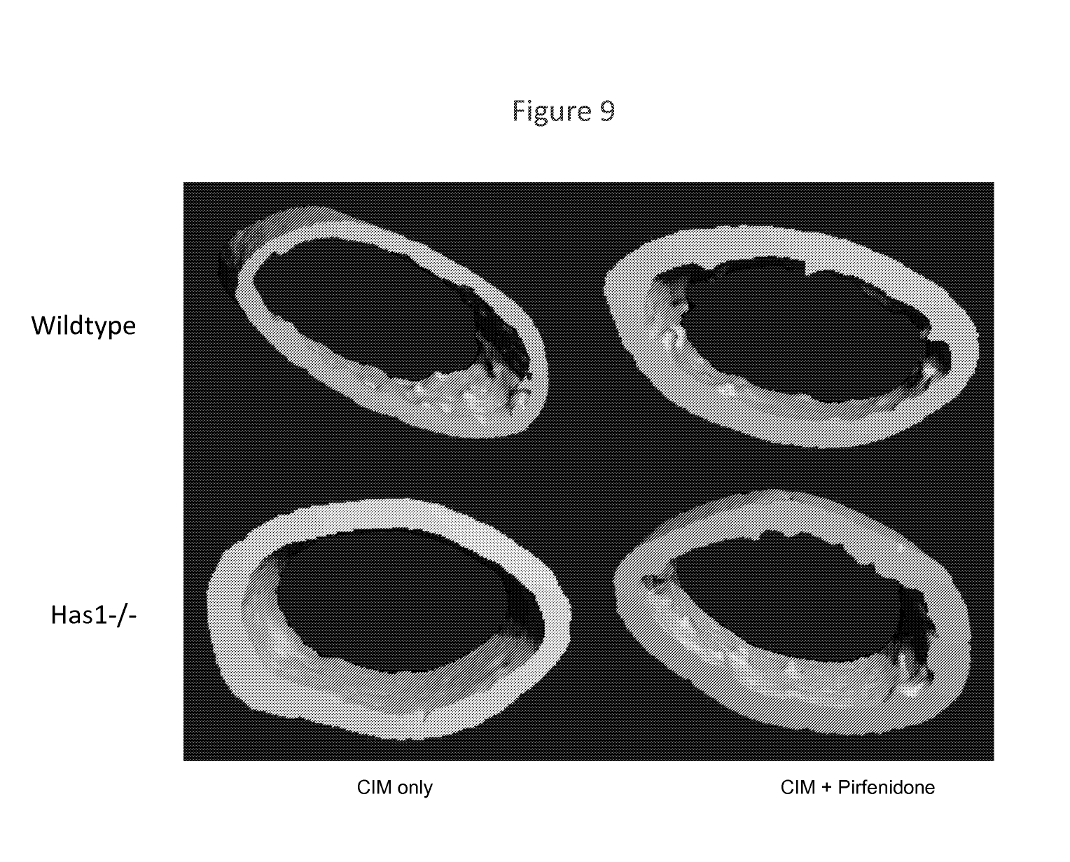

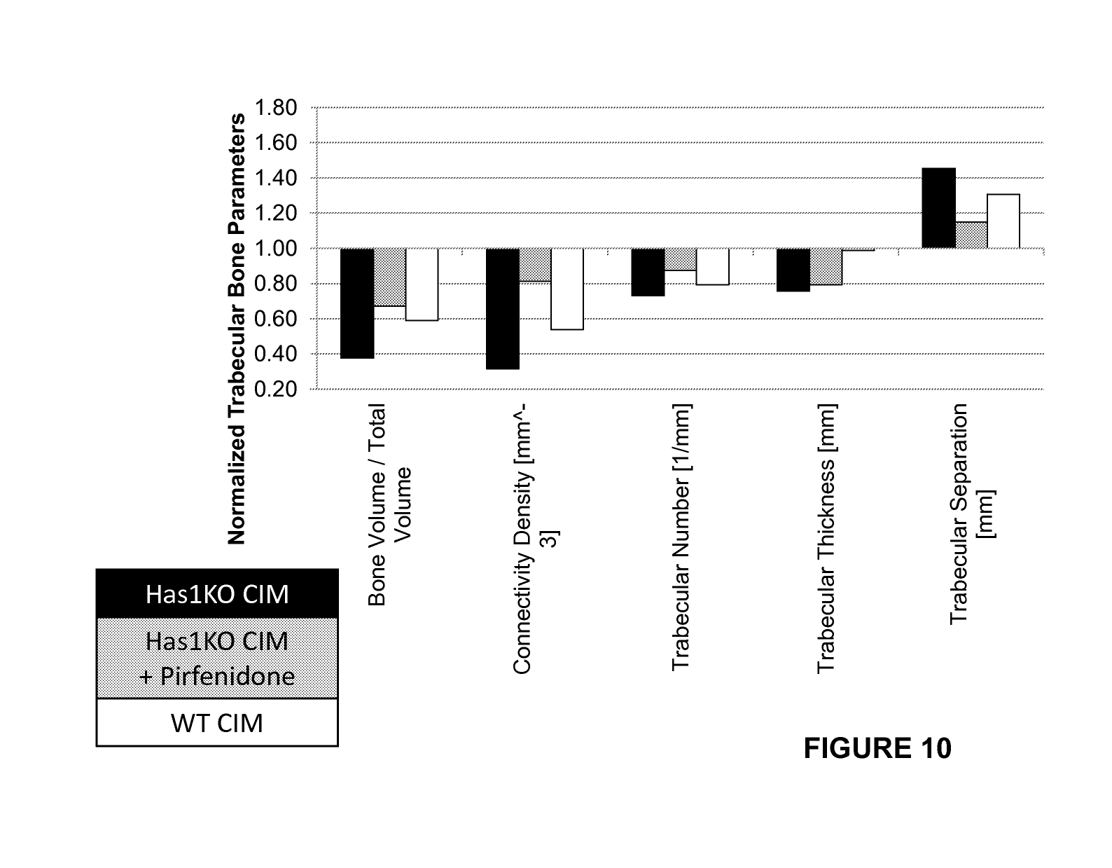

The effect of pirfenidone administration on bone structure was examined using Micro-computed Tomography (micro-CT) to identify bone differences (FIG. 7). The regions of interest examined included the trabecular bone in the femur directly posterior to the trochlear groove, where the defect was created (FIG. 8) and the cortical bone of the femoral midshaft (FIG. 9.) The trabecular bone parameters, obtained using micro-CT, indicate a recovery in injured Has1KO joints of bone-to-total volume, connectivity density, trabecular number, trabecular thickness, and trabecular separation to naive levels, surpassing even injured WT in bone recovery, and are shown in FIG. 10.

In summary, this model of joint degeneration induced by cartilage injury in the Has1KO mouse provides a novel opportunity to investigate therapeutic interventions in pre-clinical studies. The very clear response (in both histological appearance and subchondral bone morphology) to treatment with the clinically approved anti-fibrotic pirfenidone suggests its use as a murine model system in the area of therapeutic intervention for PTOA.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

We claim:

1. A method for treating or preventing the development of osteoarthritis, the method comprising administering to a subject in need of such treatment a composition comprising a therapeutically effective amount of an anti-fibrotic agent.

2. The method of claim 1, wherein the anti-fibrotic agent is selected from the group consisting of 5-methyl-1-phenylpyridin-2-one, tranilast, gamma-glutamyl transpeptidase inhibitor, fasudil, CC-930, T-5524, rosiglitazone, tocilizumab, E5564, TAK-242, GKT136901 and bosentan.

3. The method of claim 2, wherein the anti-fibrotic agent is 5-methyl-1-phenylpyridin-2-one.

4. The method of claim 1, wherein the composition further comprises at least one pharmaceutically acceptable carrier.

5. The method of claim 1, wherein the subject is a human subject.

6. The method of claim 1, wherein the anti-fibrotic agent at least partially normalizes the activation of at least one gene selected from the group consisting of Col1a1, Col1a2, Col2a1, Col3a1, Acan, Vcan, Has1, Has2, Has3, Itih2, and Tnfaip6.

7. The method of claim 6, wherein the anti-fibrotic agent at least partially normalizes the activation of at least two genes selected from the group consisting of Col1a1, Col1a2, Col2a1, Col3a1, Acan, Vcan, Has1, Has2, Has3, Itih2, and Tnfaip6.

8. The method of claim 1, wherein the anti-fibrotic agent at least partially normalizes the activation of at least one gene selected from the group consisting of the NF-κb pathway genes shown in FIG. 5A.

9. The method of claim 1, wherein the anti-fibrotic agent at least partially normalizes the activation of at least one gene selected from the group consisting of the fibrosis pathway genes shown in FIG. 5B.

10. The method of claim 1, wherein the composition is administered orally.

11. The method of claim 1, wherein the composition is administered by a route selected from the group consisting of the subcutaneous, intra-articular, intradermal, intravenous, intraperitoneal and intramuscular routes.

12. The method of claim 1, wherein the subject is a human subject having an injury to a joint selected from the group consisting of the knee, shoulder, hip, elbow, temporomandibular or ankle joints, or a joint of the hand, foot and spine.

13. The method of claim 1, wherein the subject is a human subject having a traumatic injury.

14. A method for treating an injury to a joint of a human or veterinary subject, comprising:
determining mRNA expression levels of a plurality of genes expressed in a tissue sample taken from an intra-articular region of the joint, the plurality of genes comprising at least the genes listed in FIG. 4;
calculating a reparative index score based on the mRNA expression levels of the plurality of genes, wherein the reparative index score is indicative of the quality of the repair response, and
administrating a the anti-fibrotic agent is selected from the group consisting of 5-methyl-1-phenylpyridin-2-one, tranilast, gamma-glutamyl transpeptidase inhibitor, fasudil, CC-930, T-5524, rosiglitazone, tocilizumab, E5564, TAK-242, GKT136901 and bosentan, wherein the anti-fibrotic agent is administrated depending on the reparative index score.

15. The method of claim 14, wherein the anti-fibrotic agent is 5-methyl-1-phenylpyridin-2-one.

16. The method of claim 14, wherein the tissue sample comprises material selected from the group consisting of synovial fluid, blood, cartilage, synovium, meniscal tissue, joint capsule lining, ligaments and combinations of at least two of these materials.

17. The method of claim 14, wherein calculating the reparative index score comprises: comparing the mRNA expression levels with first standard expression levels of the plurality of genes and second standard expression levels of the plurality of genes, wherein the first standard expression levels are indicative of a reparative profile and wherein the second standard expression levels are indicative of a non-reparative profile; and
determining the reparative index score based on relative values of the mRNA expression levels, the first standard expression levels and the second standard expression levels.
